(19) United States
(12) Patent Application Publication
(10) Pub. No.: US 2002/0031559 A1
(43) Pub. Date: Mar. 14, 2002

(54) Herbal suppositories

(76) Inventors: Kin C. Liang, Covina, CA (US); Liyin Liang, Covina, CA (US)

Correspondence Address: SHELDON & MAK, Suite 900, 225 South Lake Avenue, Pasadena, CA, 91101, US

(21) Appl. No.: 09/974,452

(22) Filed: Oct. 9, 2001

Related U.S. Application Data
(63) Continuation of application No. 09/520,978, filed on Mar. 8, 2000.

(51) Int. Cl. A61K 35/78

(52) U.S. Cl. 424/725

(57) ABSTRACT

A suppository for treating human ailments comprising at least one herb and a suppository vehicle. A method of treating undesired symptoms from allergic rhinitis, sinusitis, nasal congestion, nasal dripping, nasal polyps, infections, fevers, coughs, spasms, dizziness, convulsions in a human uses a suppository having herbs. Methods of producing, administrating and formulating herbal medicines in the form of suppositories to treat human aliments and disease are disclosed in this invention.

### RELATED APPLICATION

This application is a continuation application to the U.S. patent application Ser. No. 09/520,978, filed on Mar. 8, 2000.

### FIELD OF THE INVENTION

This invention is in the field of administration of a medicinal herbal suppository, and, in the field of practicing and preparation of herbal suppositories that comprise various herbs.

### BACKGROUND OF THE INVENTION

A suppository is a solid-form dosage that acts as a carrier to deliver medicine to a human body. Suppositories come with varying weights and shapes. They are applied via insertion into various body cavities such as rectum, vagina, urethra and nasal. When inserted, the suppository dissolves and releases the medicine. The medicine is mixed with body fluids. The fluids carry the medicine to mucous membrane tissues, which then absorb the medicine. The medicine can either have localized effects that treat the disease near where the suppository is applied, or systemic effects that treat the disease at other parts of the body.

Suppositories are particularly useful in administering medicine to very young and very old patients. A common suppository is approximately two grams each and is tapered at one or both ends for easy insertions. Suppositories used by children are approximately half the size and weight of the adult suppositories. There are several types of suppositories that can be found today (see U.S. Pat. No. 5,002,771; U.S. Pat. No. 4,268,501; U.S. Pat. No. 4,698,359; and U.S. Pat. No. 4,250,169).

Administration of a medicine through the digestive tract of the body could cause a breakdown in the medicinal properties of the medicine. This is because the acidic environment and enzymatic activity of the stomach and intestines often interact with the medicine and breaks down the medicinal properties. The portal circulation that takes the medicine to the liver metabolism also breaks down the effectiveness of the medicine. Thus, in order for the effective strength of the medicine to stay the same, higher dosages must be taken to make up for these degradations. Since most Western medicines possess undesirable side-effects, it is usually not recommended for taking too much medicine at a single time. In most cases the amount of medicine taken in suppository form is less than the amount needed for the same effect taken orally. Administrating a medicine using a suppository is a preferred method for the medicine's medicinal properties to be directly absorbed into the bloodstream, thereby bypassing the digestive tract to avoid such degradations.

Besides bypassing the digestive tract in administrating a medicine, there are several other advantages to the use of suppositories as a medicine delivery vehicle. For example, the amount of time necessary for the medicine to have an effect is less since the medicine is directly absorbed into the bloodstream through the mucous membrane tissues. Suppository-form medicines can easily eliminate any irritation to the stomach, which is caused by many common medicines. The use of suppositories also makes it easier to administrate medicines to patients who are young, debilitated, comatose, or otherwise unable or unwilling to swallow medicines. Suppositories are also useful for treating patients with nausea, vomiting, and other gastrointestinal disturbances.

The use of suppositories dates back to the early Egyptian, Greek and Roman Times. Currently the most common medicines found in suppository forms are Western medicines.

Oriental medicines (Chinese medicines or herbal medicines) have used medicinal herbs safely and effectively to treat many ailments for thousands of years. Presently, there are several common ways to administrate herbal medicines. The classical way is to merge selected herbs into water or other solvents such as alcohol, then simmer the herbs to make a herbal tonic tea. The more modern way to administrate the herbal medicines is to package the herbs in the forms of pills, tablets, and capsules. The latter method eliminates the bad taste of the medicine. Both are effective; however, large doses need to be taken to achieve the effective result.

The first method is abhorrent to many because of its extreme bad taste and its inconvenience in preparation and portability. Although more preferable than the first, taking herbs in pill form, tablet form, and capsule form is also abhorrent to many because some are unable to swallow so the large number of pills needed for one dose. Therefore, there is a need to have a herbal medicine delivery method that is effective and practical without a long and tedious preparation process, large quantities to consume or bad taste.

Putting the herbal medicine in a suppository form does combine the advantages of herbal medicine with the ease and efficiency of administering medicine thorough a suppository. Herbal suppositories are the answer that brings together the effectiveness and safety of herbal medicines plus the efficiency and advantages of using suppositories.

### SUMMARY OF THE INVENTION

The invention is a suppository containing at least one herb.

In one embodiment of the invention, a method of treating undesired symptoms from allergic rhinitis, sinusitis, nasal congestion, nasal dripping, and nasal polyps in a human comprising administering to the human in need thereof a nasal suppository having herbs. The amount administered is between approximately 3 mg/kg/day and approximately 28 mg/kg/day of the nasal suppository. The amount administered is between approximately 14 mg/kg/day and approximately 82 mg/kg/day of the rectal suppository. The term mg/kg/day means milligram per kilogram human body weight per day.

In other embodiment of the invention, a diaphoretic herbal mixture in a suppository to treat a human for infections in his upper respiratory tract, the herbal mixture comprising at least one herb selected from the group consisting of Fu-ling (Poria Cocos), Qian Shi (Euryale Ferox), Bi Xie (Dioscorea), Shan Yao (Dioscorea Batata) and Tian Huan Fen (Trichosanthes Kcrilowii); at least one herb selected from the group consisting of Bing Pian (Dryobalanops Aromatica), Bo He (Mentha Arvensis) and Zhang Nao (Cinnamomum Camphora); and at least one herb selected from the group consisting of Ma Huang (Ephedra sinica), Gui Zhi (Cinnamomum Cassia), Zi Su(Perilla Frutescens), Jing Jie (Schizonepeta Tenuifolia), Fang Feng (Siler Divaricatum), Qiang Huo (Notopteygium Incisium), Bai Zhi (Angelica Dahurica), Gao Ben (Ligusticum Sinense), Cang Er Zi (Xanthium Strumarium), Xin Yi Hua (Magnolia Liliflora), Bo He (Mentha Arvensis), Niu Bang Zi (Arctium Lappa), Chan Tui (Cryptotympana Atrata), Dan Dou Chi (Glycine Max), Ju Hua (Chrysanthemum Indicum), Man Jing Zi (Vitex Rotundifolia), Ge Gen (Pueraria Pseudohirsuta), Chai Hu (Bupleurum Chinense), and Sheng Ma (Cimicifuga Foetida).

### DETAILED DESCRIPTION

The following discussion describes in detail one embodiment of the invention and several variations of that embodiment. His discussion should not be construed, however, as limiting the invention to those particular embodiments. Practitioners skilled in the art will recognize numerous embodiments as well.

The present invention is a method of treating undesired symptoms from ailments or diseases in human using herbal suppositories and various suppository formulas for different diseases.

An herbal suppository is used in various body cavities such as the rectum, vagina, urethra, and nasal cavity to treat aliments or the symptoms caused by such aliments. The suppository consists of an herbal formula mixture of at least one herb and then suspending the resultant mixture in a pharmaceutically acceptable suppository.

The method of preparing an herbal suppository comprises two parts: a suppository vehicle and a pure herbal medicine. A suppository vehicle is a suppository base which holds the herbs together to form a suppository. A prepared herbal medicine can be dispersed in an oil-based or an aqueous suppository vehicle. Then the mixture is formed into a suppository form.

In herbal suppository preparation, standard traditional herbal formulas can be obtained in commercially concentrated extracts. This is made in the method that is described in U.S. Pat. Nos. 5,503,810 and 5,466,454. If a selected formulation is used, then a unique combination of herbs can be placed together and a concentrated extract can be obtained using standard methods of extraction and condensation disclosed in U.S. Pat. Nos. 5,503,810 and 5,466,454. Other methods of extracting concentrated herbal extracts can also be used for obtaining the extracts. The method of obtaining herbal extracts is not critical for this invention.

Two types of suppository bases can be used in the present invention. One is aqueous base that has hydrophilic properties and the other is oleaginous base that has hydrophobic properties. The fatty-acid based (oleaginous base) suppositories used in the present invention include materials that are commonly used for making suppositories, such as various oils and lipids mineral oils and/or higher fatty acids. Examples of these materials are cocoa butter, olive oil, soybean oil, vegetable oil, other hydrogenate fatty acids, petrolatum, various forms of paraffin, stearic acid and oleic acid. The above oils, mineral oils, and fatty acids can be either used by themselves or in combination with another to produce a suppository base possessing the desired hardness and melting point.

The aqueous base suppositories that can be used in the present invention include polymers of ethylene oxide, a.k.a. polyethylene glycols (PEG). These polymers of PEG vary in molecular weight, from 200-8000 g/mol. Also as the molecular weight of PEG increases, the hardness of PEG also increases. By combining different proportions of PEG polymers, a suppository base can be made to desired consistency and characteristics. Glycerinated gelatin is also used in this type of suppositories, but mostly as the base for vaginal suppositories. Other types of bases can also be used, such as Polysorbate. The aqueous base can be used alone or used in combination with other base materials to produce suppositories of varying consistency and solubility.

A typical embodiment of the invention, each suppository, aqueous or oil-based, has up to approximately 50% by weight of pure herbs in a pharmaceutically acceptable suppository vehicle.

In a preferred embodiment of the invention, the suppositories also contain other additives, such as stabilizers, surface active agents, stiffening agents, buffering agent, and pH adjusting agents. Examples of these additives are polysorbates, tween, and polyoxyl sterates as surface active agents; sodium sterate, spermaceti as stiffening agents; dextrose and sodium chloride as buffering agents; and sodium bicarbonate and sodium phosphate as pH adjusting agents. The types of additives and their concentrations used in a particular suppository depend upon the particular herbal formula and the suppository vehicle used.

The following is a typical hydrophilic suppository mixture (the aqueous base suppository) ingredients that make a batch of suppositories:

| Composition | Amount | | |
|---|---|---|---|
| PEG₃₃₅₀ | approx. | 15 | g |
| PEG₁₀₀₀ | approx. | 32 | g |
| PEG₂₀₀ | approx. | 3 | g |
| 5% Dextrose | approx. | 5 | cc |
| Menthol | approx. | 0.2 | g |
| Herbal Extract(s) | approx. | 35 | g |

PEG comes with various molecular weights. The molecular weights are indicated by the subscript number next to the PEG entries.

The steps of making the above batch of suppositories are:

Step 1. Add all PEGs to a heat-proof vessel or container, and proceed to increase heat to approximately 45 degrees Celsius until PEGs are completely melted;

Step 2. Add 5% dextrose and mix;

Step 3. Test pH and adjust pH by adding and mixing sodium bicarbonate and sodium phosphate until desired pH of 7 is reached;

Step 4. Add the herbal extract(s) in increments and mix until it becomes a homogeneous suspension;

Step 5. Allow the mixture to cool until approximately 36-39 degrees Celsius; and

Step 6. Pour the mixture into pre-prepared suppository shells, then place them in a cold storage.

The following is a typical hydrophobic suppository mixture (the oleaginous base suppository) ingredients that make a batch of suppositories:

| Composition | Amount | | |
|---|---|---|---|
| Cacao Butter | approx. | 38 | g |
| Yellow Wax | approx. | 5 | g |
| Petroleum Jelly | approx. | 5 | g |
| Sodium Stearate | approx. | 2 | g |
| Herbal Extract(s) | approx. | 50 | g |

The steps of making the above batch of suppositories are:

Step 1. Add cacao butter, yellow wax, and petroleum jelly in a heat-proof vessel or container and heat the temperature to approximately 60 degrees Celsius until the materials are completely melted;

Step 2. In a vessel or container, mix the sodium stearate and the herbal extract(s) thoroughly together to make a sodium stearate-herbal extract mixture;

Step 3. Slowly add the sodium stearate-herbal extract mixture in increments into the heat-proof vessel and mix thoroughly with the materials in the heat-proof vessel until they become a homogenized mixture; and

Step 4. Pour the homogenized mixture into pre-prepared suppository shells, then place them in a cold storage.

One embodiment of the invention provides a method of treating undesired symptoms from allergic rhinitis, sinusitis, nasal congestion, nasal dripping, and nasal polyps using an herbal suppository. By using different herbal formulas, the suppository form of herbal medicines can also treat other aliments and diseases.

The suppository herbal medicines can be delivered through various body cavities. However, different dosages are used in different body cavities delivery. For example, the dosages for nasal suppository are between approximately 3 mg/kg/day and approximately 28 mg/kg/day. The dosages for rectal between approximately 14 mg/kg/day and approximately 82 mg/kg/day.

A nasal suppository can also be considered as a form of a lozenge, a cachet, a solution, a suspension, an emulsion, a powder, an aerosol, a suppository, a spray, a pastille, an ointment, a cream, a paste, a foam, a gel, a tamport, or a pessary.

The herb names appeared herein are in Chinese translations and Latin. The words in the parentheses are Latin names of the herbs. These herbs and their names can be found in most herbal handbooks in local libraries.

In one embodiment of the invention, a diaphoretic herbal mixture in a suppository to treat a human for infections in his or her upper respiratory tract comprises at least one herb selected from the group consisting of Fu-ling (Poria Cocos), Qian Shi (Euryale Ferox), Bi Xie (Dioscorea), Shan Yao (Dioscorea Batata) and Tian Huan Fen (Trichosanthes Kcrilowii); at least one herb selected from the group consisting of Bing Pian (Dryobalanops Aromatica), Bo He (Mentha Arvensis) and Zhang Nao (Cinnamomum Camphora); and at least one herb selected from the group consisting of Ma Huang (Ephedra Sinica), Gui Zhi (Cinnamomum Cassia), Zi Su(Perilla Frutescens), Jing Jie (Schizonepeta Tenuifolia), Fang Feng (Siler Divaricatum), Qiang Huo (Notopteygium Incisium), Bai Zhi (Angelica Dahurica), Gao Ben (Ligusticum Sinense), Cang Er Zi (Xanthium Atrumarium), Xin Yi Hua (Magnolia Liliflora), Bo He (Mentha Arvensis), Niu Bang Zi (Arctium Lappa), Chan Tui (Cryptotympana Atrata), Dan Dou Chi (Glycine Max), Ju Hua (Chrysanthemum Indicum), Man Jing Zi (Vitex Rotundifolia), Ge Gen (Pueraria Pseudohirsuta), Chai Hu (Bupleurum Chinense), and Sheng Ma (Cimicifuga Foetida).

The herbs, Fu-ling (Poria Cocos), Qian Shi (Euryale Ferox), Bi Xie (Dioscorea), Shan Yao (Dioscorea Batata) and Tian Huan Fen (Trichosanthes Kcrilowii), are used to lower viscosity of a suspension and increase homogenization of a herbal mixture, so that they increase the ease of filling suppository. When these herbs are added in the mixture, it allows for more herbs to be added to achieve the required herb concentration while staying within the desired viscosity level. These herbs also increase the rate of absorption and effectiveness. It is found that when one or more of these herbs are mixed with other herbs, they allow us to increase the herbal weight percentage in the suppositories up to approximately 50%. It is believed that these herbs cause the surface tension between the herbs and the suppository vehicle to decrease. Therefore, a better mixture of herbs and suppository base can be made.

At least one of these herbs is required in a herbal mixture in all embodiments of this invention. On an average, the amount of these herbs used is approximately 1%-2% of the total weight of the suppository formulation.

Bing Pian (Dryobalanops Aromatica) is an herb that opens up the inner passages. It brings alertness and energy. This herb is used in medicines which treat diseases involving weakness, fatigue, chills, etc. It is used to bring the body system to a mild and temperate condition. This herb is most effective when used to achieve the following effects: facilitating the qi mechanisms, aromatically opening the orifices, breaking up phlegm, rescuing the devastated yang, invigorating the channels, invigorating the collaterals, dislodging phlegm, invigorating the blood, strengthening the transporting functions of the stomach, releasing the muscle layer, relieving tetany or spasms, relieving pain, removing painful obstructions, spreading qi and penetrating membrane sources, regulating blood, clearing the brain, dispelling wind-dampness, dispelling blood stasis, scattering or dispersing cold, overcoming dampness, raising yang, reviving from fainting, adjusting the qi, unblocking areas of stagnation, warming the menses, warming the interior, promoting the movement of qi, reviving the spirit, disseminating lung qi, augmenting qi, improving spleen, transporting function, stimulating yang, etc.

Bo He (Mentha Arvensis) is used in increasing effectiveness and action. This herb is used to cool down the body system. It is most effective when used to achieve the following effects: facilitating qi mechanism, aromatically opening the orifices, breaking up phlegm, dislodging phlegm, Strengthening transporting functions of the stomach, causing fire to descend, relieving toxicity, releasing the muscle layer, relieving tetany or spasms, relieving summer heat, relieving pain, removing painful obstructions, spreading qi and penetrating the membrane sources, regulating qi, regulating blood, cooling blood, anchoring yang, clearing the brain, clearing the sensory orifices, clearing heat, gently dispersing dryness, clearing dryness, dispelling wind-dampness, dispelling cold, dispelling blood stasis, dispersing fire from constraint, dispersing wind, spreading liver qi, reviving from fainting, adjusting qi, unblocking areas of stagnation, extinguishing wind, promoting the movement of qi, reviving the spirit, disseminating lung qi, controlling spasms and convulsions, sedating spirit, stopping coughing, alleviating dysenterica disorders, alleviating pain, driving out water, and driving out phlegm.

Zhang Nao (Cinnamomum Camphora) is used in increasing effectiveness and action. This herb is used to warm up the body system. It is most effective when trying to achieve the following effects: facilitating qi mechanisms, aromatically opening the orifices, supporting the normal qi, securing and binding, breaking up phlegm, restoring yang, rescuing the devastating yang, invigorating the channels, invigorating the collaterals, dislodging phlegm, invigorating blood, Strengthening transporting functions of the stomach, releasing the muscle layer, relieving tetany or spasms, relieving pain, removing painful obstructions, spreading qi and penetrating to membrane sources, opening the orifices, regulating qi, regulating blood, clearing brain, dispelling wind-dampness, dispelling cold, dispelling blood stasis, scattering or dispersing cold, overcoming dampness, raising yang, reviving from fainting, adjusting qi, unblocking areas of stagnation, warming the menses, warming the interior, promoting the movement of qi, reviving the spirit, disseminating lung qi, augmenting qi, improving spleen transporting functions, stimulating yang, controlling spasms and convulsions, sedating spirit, stopping coughing, alleviating dysenterica disorders, alleviating pain, driving out water, and driving out phlegm.

Regarding the herbs Bing Pian (Dryobalanops Aromatica), Bo He (Mentha Arvensis) and Zhang Nao (Cinnamomum Camphora), at least one of these herbs is typically required in all embodiments of this invention. On an average, the amount of these herbs used in this invention varies from 0.2%-0.3% by weight.

The diaphoretic formulas are used for relieving the infections in the upper respiratory tract and acute infectious disease. Most of the diaphoretic are febrifugal, anti-viral, and anti-bacterial.

In another embodiment of the invention, an antipyretic herbal mixture in a suppository to treat a human for alleviating heat symptoms comprises the herbs discussed previously and at least one herb selected from the group consisting of Shi Gao (Gypsum Fibrosum), Zhi Mu (Anemarrhena), Lu Gen (Phragmites Communis), Tian Hua Fen (Trichosanthes Kirilowii), Zhu Lu (Phyllostachys Nigra), Shen Zhi Zi (Gardenia Jasminoides), Xia Ku Cao (Prunella Vulgaris), Huang Qin (Scutellaria Baicalensis), Huang Lian (Coptis Teeta), Huang Bai (Phellodendron Amurense), Long Dan Cao (Gentiana Scabra), Sheng Di Huang (Rehmannia Glutinosa), Xuan Shen (Scrophulairia Ningpoensis), Mu Dan Pi (Paeonia Suffruticosa), Chi Shao (Paeonia Veitchii Lynch), Zi Cao (Macrotomia Euchroma), Jin Yin Hua (Lonicera Japonica), Lian Qiao (Forsythia Suspensa), Pu Gong Ying (Taraxacum Mangolicum), Zi Hua Di Ding (Viola Yedoensis), Da Qing Ye (Isatis Tinctoria), Niu Huang (Bos Taurus), Tu Fu Ling (Similax Glabra), Yu Xing Cao (Houttuynia Cordata), She Gan (Belamcanda Chinensis), Shan Dou Gen (Sophora Subprostrata), Bai Hua She She Cao (Hedyotis diffusa), Xiong Dan (Selenarctos Thibetanus), Bai Xian Pi (Dictamnus Dasycarpus), Lu Dou Yi (Phaseolus Mungo), Zing Hao (Artemisia Apiacea), Di Gua Pi (Lycium Chinense), Yin Chai Hu (Stellaria Dichotoma), and Hu Huang Lian (Picrorrhiza Kurroa).

The antipyretic herbal formulas in this category are usually cold or cool in nature. They are used to alleviate heat symptoms. These herbs have anti-viral, anti-fungal and anti-bacterial, immunity increasing, Antipyretic, anti-hypertensive, and sedative pharmaceutical actions.

In one embodiment of the invention, an expectorant, antitussive, and anti-asthmatic herbal mixture in a suppository form to eliminate cold and heat type phlegm by increasing or decreasing blood flow to the lung and to suppress coughing and asthma by dilating the bronchi and the respiratory tract comprises the herbs discussed previously and at least one herb selected from the group consisting of Ban Xia (Pinellia Ternate), Jie Geng (Platycodon Grandiflorum), Xuan Fu Hua (Inula Japonica), Bai Qian (Cynanchum Stauntoni), Qian Hu (Peucedanum Praeruptorum), Gua Lo Ren (Trichosanthes Kirilowii), Chuan Bei Mu (Fritillaria Cirrhosa), Tian Zhu Huang (Phyllostachys Reticulata), Hai Zao (Sargassum Fusiforme), Xing Ren (Prunus Armeniaca), Bai Bu (Stemona Sessilifolia), Zzi Wan (Aster Tataricus), Ting Li Zi (Lepidium Apetalum), Pi Pa Ye (Eriobotrya Japonica).

The Expectorant herbal formulas are the formulas that facilitate coughing in productive coughs by thinning the sputum. Some can also increase the secretion in the respiratory tract. They can dilate the bronchial smooth muscles, and relieve the tracheal spasms. There are generally four types of expectorants, one that eliminates the cold phlegm, one that eliminates the heat phlegm, one that is antitussive and anti-asthmatic, and one that relieves coughs through moistening effects. Such formulas can also be used as antitussive and anti-asthmatic agent.

In one embodiment of the invention, a warm-type herbal mixture in a suppository to treat a human for interior cold syndromes comprises the herbs discussed previously and at least one herb selected from the group consisting of Fu Zi (Aconitum Carmichaeli), Gan Jiang (Zingiber Rhizoma), Rou Gui (Cinnamomum Cassia), Wu Zhi Yu (Evodia Rutaecarpa), Xi Xin (Asarum Heterotropoides), Hua Jiao (Zanthoxylum Bungeanum), Ding Xiang (Eugenia Caryophyllata), Xiao Hui Xiang (Foeniculum Vulgare), Hu Jiao (Piper Nigrum), Qing Pi (Citrus Reticulata), Chen Pi (Citrus Reticulate), Mu Xiang (Saussurea Lappa), Xiang Fu Zi (Cyperus Orotundus), Zhi Shi (Citrus Aurantium), and Chuan Lian Zi (Melia Toosendan).

The warming formulas are used for interior cold syndromes. These conditions can be caused by pathogenic cold and/or interior depletion of yang energy. Such herbs have actions on the digestive tract system, action on the cardiovascular system, and actions on the nervous system. These herbs also have actions of activating and regulating qi flow, thus strengthening the digestive tract system, the cardiovascular system, and the nervous system.

In one embodiment of the invention, a digestive herbal mixture in a suppository to assist a human in increase digestion of fat, protein and carbohydrates comprises the herbs discussed previously and at least one herb selected from the group consisting of Shen Qu (Massa medicate Fermentata), Shan Zha (Crataegus Pinnatifida), Mai Ya (Hordeun Sinica), Lai Fu Zi (Raphanus Sativus), and Ji Nei Jin (Gallus Gallus Domesticus).

The digestive formulas can increase the digestion of fat, protein, and carbohydrates. They can enhance the appetite by increasing the secretion of gastric acid, digestion, and evacuation.

In one embodiment of the invention, a tonic herbal mixture in a suppository to replenish depleted qi (vital energy) and blood, and to balance yin (vital essence) and yang (vital function) in a human comprises the herbs discussed previously and at least one herb selected from the group consisting of Ren Shen (Panax Ginseng), Xi Yang Shen (Panax Quinquefolium), Dang Shen (Condonopsis Pilosula), Huang Qi (Astragalus Monggholicus), Bai Shu (Atractylodes Macrocephala), Shan Yao (Discorea Batata), Gan Cao (Glycyrrhiza Uralensis), Da Zao (Zizyphus Jujuba), Bai Bian Dou (Dolichos Lablab), Rou Cong Rong (Cistache Salsa), Xian Mao (Curculigo Orchioides), Yin Yang Huo (Epimedium Macranthum), Hu Lu Ba (Trichosanthes Kirilowii), Du Zhong (Eucommia Ulmoides), Xu Duan (Dipsacus Asper), Gou Ji (Cibotium Barometz), Gu Sui Bu (Drynaria Fortunei), Bu Gu Zhi (Psoralea Corylifolia), Yi Zhi Ren (Alpinia Oxyphylla), Dong Chong Xia Cao (Cordyceps Sinensis), Ha Jie (Gecko Gecko), Hu Tao (Juglans Regia), Tu Si Zi (Cuscuta Chinensis), Sha Yuan Ji Li (Astragalus Complanatus), Dong Gui (Angelica Senensis), Di Huang (Rehmannia Glutinosa), He Shou Wu (Polygonum Multiflorum), Bai Shao (Paeonia Lactiflora), E Jiao (Asini), Long Yin Rou (Euphoria Longan), Sha Shen (Adenophora Tetraphylla), Mai Men Dong (Ophiopogon Japonicus), Tian Men Dong (Asparagus Cochinchinensis), Shi Hu (Dendrobium Nobile), Bai He (Lilium Brownii), Guo Qi Zi (Lycium Chinense), Nu Zhen Zi (Ligustrum Lucidum), Gui Ban (Chinemys Reevesii), and Bie Jia (Amyda Sinensis).

The tonics formulas are used to replenish depletions of Qi, blood, Yin, and Yang. Such formulas have actions on the immunity of the body. It can increase the adaptability of the body. It can have action of the endocrine and the nervous system, and have action on metabolism. It can increase the productivity of the body at work.

In one embodiment of the invention, a blood regulating herbal mixture in a suppository formula to eliminate blood stasis, invigorate blood circulation and hemostatic in a human comprises the herbs discussed previously and at least one herb selected from the group consisting of Hong Hua (Carthamus Tinctorius), Chuan Xiong (Ligusticum Wallichii), Ru Xiang (Boswellia Carterii), Mo Yao (Commiphora Myrrha), Yan Hu Suo (Corydalis yunhusuo), Yu Jin (Curcuma Aromatica), Jiang Huang (Curcuma Longa), Er Shu (Curcuma Zedoaria), Jing San Ling (Sparganium Racemosum), Dan Shen (Salvia Miltiorrhiza), Yi Mu Cao (Leonurus Heterophyllus), Ji Xue Teng (Mucuna Birdwoodiana), Tao Ren (Prunus Persica), Wu Ling Zhi (Trogopterus Xanthipes), Huai Niu Xi (Achyranthes Bidentata), Ze Lan (Lycopus Lucidus), Wang Bu Liu Xing (Vaccaria Segetalis), Di Yu (Sanguisorba Officinalis), Da Ji (Cirsium Japonicum), Bai Mao Gen (Imperata Cylindrical), Huai Hua (Sophora Japonica), San Qi (Panax Pseudoginseng), Qian Cao Gen (Rubia Cordifolia), Ai Ye (Artemisia Argyi), and Xian He Cao (Agrimonia Pilosa).

The blood regulating formulas are used to eliminate blood stasis. Such herbs can be used in circulation, in inflammation, for metastasises, for soft tissue injures, and to regulate the immune system. They also shorten the process of coagulation, constrict the local blood vessels and inhibit fibrinolysis.

In one embodiment of the invention, a resuscitating herbal mixture in a suppository formula to treat symptoms of spasm, dizziness, and convulsion of a human comprises the herbs discussed previously and at least one herb selected from the group consisting of Zhen Zhu Mu (Pteria Martensii), Bing Pian (Dryobalanops Aromatica), Shi Chang Pu (Acorus Gramineus), Su He Xiang (Liquidambar Orientalis), Shi Jue Ming (Haliotis Ovina), Mu Li (Ostrea Gigas), Zhen Zhu (Pteria Martensii), Gou Teng (Uncaria Rhyncho Phyla), and Tian Ma (Gastrodia Elata).

The resuscitating formulas can be used to treat symptoms of spasms, dizziness, and convulsions caused by rebellious qi due to liver and kidney yin deficiency with ascendant liver yang. These herbs have actions of increasing liver and kidney yin and decreasing liver yang. They are categorized as anti-hypertensive, anti-convulsant, anti-epileptic, sedative and hypnotic.

In one embodiment of the invention, an astringent herbal mixture in a suppository formula being able to coagulate with blood and to block injured capillaries thus helping to stop bleeding, to regulate smooth muscles of urinary-genital tracts, digestive tracts, respiratory tracts and blood vessels inhibiting secretion of mucous and sweat glands, and to ease cough, diarrhea bleeding spermatorrhoea, excessive urination and perspiration comprises the herbs discussed previously and at least one herb selected from the group consisting of Wu Wei Zi (Schisandra Chinensis), Wu Mei (Prunus Mume), Wu Bei Zi (Schlechtendalia Chinensis), Ron Dou Kou (Myristica Fragrans), Lian Zi (Nelumbo Nucifea), Qian Shi (Euryale Ferox), Fu Pen Zi (Rubus Palmatus), and Shan Zhu Yu (Comus Officinalis).

The astringents formulas contains tannin organic acids or inorganic salts. Such formulas can form a protective layer from foreign toxins when in contact with the mucosa. It can also coagulate with blood and stop bleeding.

In one embodiment of the invention, a purgative herbal mixture in a suppository to act as an anti-viral, anti-bacterial and anti-fungal agent in a human comprises the herbs discussed previously and at least one herb selected from the group consisting of Da Huang (Rheum Palmatum), Lu Hui (Aloe Vera), Huo Ma Ren (Cannabis Sativa), Yu Li Ren (Prunus Ja Ponica), Yuan Hua (Daphne Genkwa), Ba Dou (Croton Tigilium), and Da Ji (Euphorbia Pekinensis).

The purgative formulas will cause purgative or emollient action. They are cathartics and anti-viral, anti-bacterial, and anti-fungal agents.

In one embodiment of the invention, a diuretic herbal mixture in a suppository include diuretics, aromatic herbs and anti rheumatic to increase the rate of urine flow and to reduce dampness in a human to use in bi-syndrome comprises the herbs discussed previously and at least one herb selected from the group consisting of Du Huo (Angelica Pubescens), Can Sha (Bombyx Mori), Wei Ling Xian (Clematis Chinensis), Fang Ji (Stephania Tetrandra), Qin Jiao (Gentiana Macrophylla), Xi Qian Cao (Siegesbeckia Pubescens), Mu Gua (Chaenomeles Lagenaria), Huo Ji Sheng (Viscum Coloratum), Wu Jia Pi (Acanthopanax Gracilistylus), Hai Tong PI (Erythrina Indica), Cang Su (Atractylodes Lancea), Hou Pu (Magnolia Officinalis), Huo xiang (Pogosstemon Cablin), Sha Ren (Amomun Xanthioides), Fu-ling (Poria Cocos), Ze Xie (Alismatis Orientalis), Yi Yi Ren (Coix Lachymajajabi), Che Qianzi (Plantago Asiatica), Mu Tong (Clematis Armandi), Bi Xie (Dioscorea), and Bian Xu (Polygonum Aviculare).

The diuretic herbal formulas can increase the rate of urine flow and resolve dampness. There are increased glomeralar filtration or decreased tubular reabsorption. The most effective diuretics influence the excretion of sodium ion rather than the water itself.

In one embodiment of the invention, a sedative herbal mixture in a suppository to calm a human and induce sleeping status comprises the herbs discussed previously and at least one herb selected from the group consisting of Zhu Sha (Cinnabaris), Ci Shi (Magnetium), Suan Zao Ren (Zizyphus Sativavar), Bai Zi Ren (Biotae Orientalis Semen), Yuan Zhi (Polygala Tenuifolia), and He Huan Pi (Albizza Julivrissin).

The sedative formulas are agents used to calm or induce sleeping status. Tranquilizers imply mental calmness without depression of mental activity or alertness. There are two kinds of sedatives; heavy and herbal nourish sedatives.

Having thus described the invention, it should be apparent that numerous modifications and adaptations may be resorted to without departing from the scope and fair meaning of the instant invention as set forth hereinabove and as described hereinbelow by the claims.

What is claimed is:

1. A suppository for treating human ailments comprising at least one herb and a suppository vehicle.

2. The suppository of claim 1 wherein the suppository vehicle is a suppository base which holds the herb together to form a suppository.

3. The suppository of claim 1 wherein the herb comprises at least 50% by weight of the suppository.

4. A method of treating undesired symptoms from allergic rhinitis, sinusitis, nasal congestion, nasal dripping, nasal polyps, infections, fevers, coughs, spasms, dizziness, convulsions in a human comprising administering to the human in need thereof a suppository having herbs.

5. The method of claim 4 wherein the administration is a body cavity selected from the group consisting of nasal, rectum, vagina, and urethra.

6. The method of claim 4 wherein the administration is nasal.

7. The method of claim 4 wherein the amount of herbs administered is between approximately 3 mg/kg/day and approximately 28 mg/kg/day in the nasal suppository.

8. The method of claim 4 wherein the amount of herbs administered is between approximately 14 mg/kg/day and approximately 82 mg/kg/day in the rectal suppository.

9. The method of claim 4 wherein the undesired symptoms comprise nasal congestion, and nasal dripping.

10. The method of claim 4 wherein the nasal suppository is administrated in the form of a lozenge, a cachet, a solution, a suspension, an emulsion, a powder, an aerosol, a suppository, a spray, a pastille, an ointment, a cream, a paste, a foam, a gel, a tamport, or a pessary.

11. A diaphoretic herbal mixture in a suppository to treat a human for infections in his upper respiratory tract, the herbal mixture comprising:
a. at least one herb selected from the group consisting of Fu-ling (Poria Cocos), Qian Shi (Euryale Ferox), Bi Xie (Dioscorea), Shan Yao (Dioscorea Batata) and Tian Huan Fen (Trichosanthes Kcrilowii);
b. at least one herb selected from the group consisting of Bing Pian (Dryobalanops Aromatica), Bo He (Mentha Arvensis) and Zhang Nao (Cinnamomum Camphora); and
c. at least one herb selected from the group consisting of Ma Huang (Ephedra Sinica), Gui Zhi (Cinnamomum Cassia), Zi Su(Perilla Frutescens), Jing Jie (Schizonepeta Tenuifolia), Fang Feng (Siler Divaricatum), Qiang Huo (Notopteygium Incisium), Bai Zhi (Angelica Dahurica), Gao Ben (Ligusticum Sinense), Cang Er Zi (Xanthium Strumarium), Xin Yi Hua (Magnolia Liliflora), Bo He (Mentha Arvensis), Niu Bang Zi (Arctium Lappa), Chan Tui (Cryptotympana Atrata), Dan Dou Chi (Glycine Max), Ju Hua (Chrysanthemum Indicum), Man Jing Zi (Vitex Rotundifolia), Ge Gen (Pueraria Pseudohirsuta), Chai Hu (Bupleurum Chinense), and Sheng Ma (Cimicifuga Foetida).

12. An antipyretic herbal mixture in a suppository to treat a human for alleviating heat symptoms, the herbal mixture comprising:
a. at least one herb selected from the group consisting of Fu-ling (Poria Cocos), Qian Shi (Euryale Ferox), Bi Xie (Dioscorea), Shan Yao (Dioscorea Batata) and Tian Huan Fen (Trichosanthes Kcrilowii);
b. at least one herb selected from the group consisting of Bing Pian (Dryobalanops Aromatica), Bo He (Mentha Arvensis) and Zhang Nao (Cinnamomum Camphora); and
c. at least one herb selected from the group consisting of Shi Gao (Gypsum Fibrosum) Zhi Mu (Anemarrhena), Lu Gen (Phragmites Communis), Tian Hua Fen (Trichosanthes Kirilowii), Zhu Lu (Phyllostachys Nigra), Shen Zhi Zi (Gardenia Jasminoides), Xia Ku Cao (Prunella Vulgaris), Huang Qin (Scutellaria Baicalensis), Huang Lian (Coptis Teeta), Huang Bai (Phellodendron Amurense), Long Dan Cao (Gentiana Scabra), Sheng Di Huang (Rehmannia Glutinosa), Xuan Shen (Scrophulairia Ningpoensis), Mu Dan Pi (Paeonia Suffruticosa), Chi Shao (Paeonia Veitchii Lynch), Zi Cao (Macrotomia Euchroma), Jin Yin Hua (Lonicera Japonica), Lian Qiao (Forsythia Suspensa), Pu Gong Ying (Taraxacum Mangolicum), Zi Hua Di Ding (Viola Yedoensis), Da Qing Ye (Isatis Tinctoria), Niu Huang (Bos Taurus), Tu Fu Ling (Similax Glabra), Yu Xing Cao (Houttuynia Cordata), She Gan (Belamcanda Chinensis), Shan Dou Gen (Sophora Subprostrata), Bai Hua She She Cao (Hedyotis Diffusa), Xiong Dan (Selenarctos Thibetanus), Bai Xian Pi (Dictamnus Dasycarpus), Lu Dou Yi (Phaseolus Mungo), Zing Hao (Artemisia Apiacea), Di Gua Pi (Lycium Chinense), Yin Chai Hu (Stellaria Dichotoma), and Hu Huang Lian (Picrorrhiza Kurroa).

13. An expectorant, antitussive, and anti-asthmatic herbal mixture in a suppository form to eliminate cold and heat type phlegm by increasing or decreasing blood flow to the lung and to suppress coughing and asthma by dilating the bronchi and the respiratory tract. The herbal mixture comprises of:
a. at least one herb selected from the group consisting of Fu-ling (Poria Cocos), Qian Shi (Euryale Ferox), Bi Xie (Dioscorea), Shan Yao (Dioscorea Batata) and Tian Huan Fen (Trichosanthes Kcrilowii);
b. at least one herb selected from the group consisting of Bing Pian (Dryobalanops Aromatica), Bo He (Mentha Arvensis) and Zhang Nao (Cinnamomum Camphora); and
c. at least one herb selected from the group consisting of Ban Xia (Pinellia Temate), Jie Geng (Platycodon Grandiflorum), Xuan Fu Hua (Inula Japonica), Bai Qian (Cynanchum Stauntoni), Qian Hu (Peucedanum Praeruptorum), Gua Lo Ren (Trichosanthes Kirilowii), Chuan Bei Mu (Fritillaria Cirrhosa), Tian Zhu Huang (Phyllostachys Reticulata), Hai Zao (Sargassum Fusifolme), Xing Ren (Prunus Armeniaca), Bai Bu (Stemona Sessilifolia), Zzi Wan (Aster Tataricus), Ting Li Zi (Lepidium Apetalum), Pi Pa Ye (Eriobotrya Japonica).

14. A warm-type herbal mixture in a suppository to treat a human for interior cold syndromes, the herbal mixture comprising:
a. at least one herb selected from the group consisting of Fu-ling (Poria Cocos), Qian Shi (Euryale Ferox), Bi Xie (Dioscorea), Shan Yao (Dioscorea Batata) and Tian Huan Fen (Trichosanthes Kcrilowii);
b. at least one herb selected from the group consisting of Bing Pian (Dryobalanops Aromatica), Bo He (Mentha Arvensis) and Zhang Nao (Cinnamomum Camphora); and
c. at least one herb selected from the group consisting of Fu Zi (Aconitum Carmichaeli), Gan Jiang (Zingiber Rhizoma), Rou Gui (Cinnamomum Cassia), Wu Zhi Yu (Evodia Rutaecarpa), Xi Xin (Asarum Heterotropoides), Hua Jiao (Zanthoxylum Bungeanum), Ding Xiang (Eugenia Caryophyllata), Xiao Hui Xiang (Foeniculum Vulgare), Hu Jiao (Piper Nigrum), Qing Pi (Citrus Reticulata), Chen Pi (Citrus Reticulate), Mu Xiang (Saussurea Lappa), Xiang Fu Zi (Cyperus Orotundus), Zhi Shi (Citrus Aurantium), and Chuan Lian Zi (Melia Toosendan).

15. A digestive herbal mixture in a suppository to assist a human in increase digestion of fat, protein and carbohydrates, the herbal mixture comprising:
a. at least one herb selected from the group consisting of Fu-ling (Poria Cocos), Qian Shi (Euryale Ferox), Bi Xie (Dioscorea), Shan Yao (Dioscorea Batata) and Tian Huan Fen (Trichosanthes Kcrilowii);
b. at least one herb selected from the group consisting of Bing Pian (Dryobalanops Aromatica), Bo He (Mentha Arvensis) and Zhang Nao (Cinnamomum Camphora); and
c. at least one herb selected from the group consisting of Shen Qu (Massa Medicate Fermentata), Shan Zha (Crataegus Pinnatifida), Mai Ya (Hordeun Sinica), Lai Fu Zi (Raphanus Sativus), and Ji Nei Jin (Gallus Gallus Domesticus).

16. A tonic herbal mixture in a suppository to replenish depleted qi (vital energy) and blood, and to balance yin (vital essence) and yang (vital function) in a human, the herbal mixture comprising:
a. at least one herb selected from the group consisting of Fu-ling (Poria Cocos), Qian Shi (Euryale Ferox), Bi Xie (Dioscorea), Shan Yao (Dioscorea Batata) and Tian Huan Fen (Trichosanthes Kcrilowii);
b. at least one herb selected from the group consisting of Bing Pian (Dryobalanops Aromatica), Bo He (Mentha Arvensis) and Zhang Nao (Cinnamomum Camphora); and
c. at least one herb selected from the group consisting of Ren Shen (Panax Ginseng), Xi Yang Shen (Panax Quinquefolium), Dang Shen (Condonopsis Pilosula), Huang Qi (Astragalus Monggholicus), Bai Shu (Atractylodes Macrocephala), Shan Yao (Discorea Batata), Gan Cao (Glycyrrhiza Uralensis), Da Zao (Zizyphus Jujuba), Bai Bian Dou (Dolichos Lablab), Rou Cong Rong (Cistache Salsa), Xian Mao (Curculigo Orchioides), Yin Yang Huo (Epimedium Macranthum), Hu Lu Ba (Trichosanthes Kirilowii), Du Zhong (Eucommia Ulmoides), Xu Duan (Dipsacus Asper), Gou Ji (Cibotium Barometz), Gu Sui Bu (Drynaria Fortunei), Bu Gu Zhi (Psoralea Corylifolia), Yi Zhi Ren (Alpinia Oxyphylla), Dong Chong Xia Cao (Cordyceps Sinensis), Ha Jie (Gecko Gecko), Hu Tao (Juglans Regia), Tu Si Zi (Cuscuta Chinensis), Sha Yuan Ji Li (Astragalus Complanatus), Dong Gui (Angelica Senensis), Di Huang (Rehmannia Glutinosa), He Shou Wu (Polygonum Multiflorum), Bai Shao (Paeonia Lactiflora), E Jiao (Asini), Long Yin Rou (Euphoria Longan), Sha Shen (Adenophora Tetraphylla), Mai Men Dong (Ophiopogon Japonicus), Tian Men Dong (Asparagus Cochinchinensis), Shi Hu (Dendrobium Nobile), Bai He (Lilium Brownii), Guo Qi Zi (Lycium Chinense), Nu Zhen Zi (Ligustrum Lucidum), Gui Ban (Chinemys Reevesii), and Bie Jia (Amyda Sinensis).

17. A blood regulating herbal mixture in a suppository formula to eliminate blood stasis, invigorate blood circulation and hemostatic in a human, the herbal mixture comprising:
a. at least one herb selected from the group consisting of Fu-ling (Poria Cocos), Qian Shi (Euryale Ferox), Bi Xie (Dioscorea), Shan Yao (Dioscorea Batata) and Tian Huan Fen (Trichosanthes Kcrilowii);
b. at least one herb selected from the group consisting of Bing Pian (Dryobalanops Aromatica), Bo He (Mentha Arvensis) and Zhang Nao (Cinnamomum Camphora); and
c. at least one herb selected from the group consisting of Hong Hua (Carthamus Tinctorius), Chuan Xiong (Ligusticum Wallichii), Ru Xiang (Boswellia Carterii), Mo Yao (Commiphora Myrrha), Yan Hu Suo (Corydalis Yunhusuo), Yu Jin (Curcuma Aromatica), Jiang Huang (Curcuma Longa), Er Shu (Curcuma Zedoaria), Jing San Ling (Sparganium Racemosum), Dan Shen (Salvia Miltiorrhiza), Yi Mu Cao (Leonurus Heterophyllus), Ji Xue Teng (Mucuna Birdwoodiana), Tao Ren (Prunus Persica), Wu Ling Zhi (Trogopterus Xanthipes), Huai Niu Xi (Achyranthes Bidentata), Ze Lan (Lycopus Lucidus), Wang Bu Liu Xing (Vaccaria Segetalis), Di Yu(sanguisorba Officinalis), Da Ji (Cirsium Japonicum), Bai Mao Gen (Imperata Cylindrical), Huai Hua (Sophora Japonica), San Qi (Panax Pseudoginseng), Qian Cao Gen (Rubia Cordifolia), Ai Ye (Artemisia Argyi) and Xian He Cao (Agrimonia Pilosa).

18. A resuscitating herbal mixture in a suppository formula to treat symptoms of spasm, dizziness, and convulsion of a human, the herbal mixture comprising:
a. at least one herb selected from the group consisting of Fu-ling (Poria Cocos), Qian Shi (Euryale Ferox), Bi Xie (Dioscorea), Shan Yao (Dioscorea Batata) and Tian Huan Fen (Trichosanthes Kcrilowii);
b. at least one herb selected from the group consisting of Bing Pian (Dryobalanops Aromatica), Bo He (Mentha Arvensis) and Zhang Nao (Cinnamomum Camphora); and
c. at least one herb selected from the group consisting of Zhen Zhu Mu (Pteria Martensii), Bing Pian (Dryobalanops Aromatica), Shi Chang Pu (Acorus Gramineus), Su He Xiang (Liquidambar Orientalis), Shi Jue Ming (Haliotis Ovina), Mu Li (Ostrea Gigas), Zhen Zhu (Pteria Martensii), Gou Teng (Uncaria Rhyncho Phyla), and Tian Ma (Gastrodia Elata).

19. An astringent herbal mixture in a suppository formula being able to coagulate with blood and to block injured capillaries thus helping to stop bleeding, to regulate smooth muscles of urinary-genital tracts, digestive tracts, respiratory tracts and blood vessels inhibiting secretion of mucous and sweat glands, and to ease cough, diarrhea bleeding spermatorrhoea, excessive urination and perspiration, the herbal mixture comprising:
a. at least one herb selected from the group consisting of Fu-ling (Poria Cocos), Qian Shi (Euryale Ferox), Bi Xie (Dioscorea), Shan Yao (Dioscorea Batata) and Tian Huan Fen (Trichosanthes Kcrilowii);
b. at least one herb selected from the group consisting of Bing Pian (Dryobalanops Aromatica), Bo He (Mentha Arvensis) and Zhang Nao (Cinnamomum Camphora); and
c. at least one herb selected from the group consisting of Wu Wei Zi (Schisandra Chinensis), Wu Mei (Prunus Mume), Wu Bei Zi (Schlechtendalia Chinensis), Rou Dou Kou (Myristica Fragrans), Lian Zi (Nelumbo Nucifea), Qian Shi (Euryale Ferox), Fu Pen Zi (Rubus Palmatus), and Shan Zhu Yu (Comus Officinalis).

20. A purgative herbal mixture in a suppository to act as an anti-viral, anti-bacterial and anti-fungal agent in a human, the herbal mixture comprising:
a. at least one herb selected from the group consisting of Fu-ling (Poria Cocos), Qian Shi (Euryale Ferox), Bi Xie (Dioscorea), Shan Yao (Dioscorea Batata) and Tian Huan Fen (Trichosanthes Kcrilowii);
b. at least one herb selected from the group consisting of Bing Pian (Dryobalanops Aromatica), Bo He (Mentha Arvensis) and Zhang Nao (Cinnamomum Camphora); and
c. at least one herb selected from the group consisting of Da Huang (Rheum Palmatum), Lu Hui (Aloe Vera), Huo Ma Ren (Cannabis Sativa), Yu Li Ren (Prunus Ja Ponica), Yuan Hua (Daphne Genkwa), Ba Dou (Croton Tigilium), and Da Ji (Euphorbia Pekinensis).

21. A diuretic herbal mixture in a suppository include diuretics, aromatic herbs and anti rheumatic to increase the rate of urine flow and to reduce dampness in a human to use in bi-syndrome, the herbal mixture comprising:
a. at least one herb selected from the group consisting of Fu-ling (Poria Cocos), Qian Shi (Euryale Ferox), Bi Xie (Dioscorea), Shan Yao (Dioscorea Batata) and Tian Huan Fen (Trichosanthes Kcrilowii);
b. at least one herb selected from the group consisting of Bing Pian (Dryobalanops Aromatica), Bo He (Mentha Arvensis) and Zhang Nao (Cinnamomum Camphora); and
c. at least one herb selected from the group consisting of du Huo (Angelica Pubescens), Can Sha (Bombyx Mori), Wei Ling Xian (Clematis Chinensis), Fang Ji (Stephania Tetrandra), Qin Jiao (Gentiana Macrophylla), Xi Qian Cao (Siegesbeckia Pubescens), Mu Gua (Chaenomeles Lagenaria), Huo Ji Sheng (Viscum Coloratum), Wu Jia Pi (Acanthopanax Gracilistylus), Hai Tong Pi (Erythrina Indica), Cang Su (Atractylodes Lancea), Hou Pu (Magnolia Officinalis), Huo Xiang (Pogosstemon Cablin), Sha Ren (Amomun Xanthioides), Fu-ling (Poria Cocos), Ze Xie (Alismatis Orientalis), Yi Yi Ren (Coix Lachmajajabi), Che Qianzi (Plantago Asiatica), Mu Tong (Clematis Armandi), Bi Xie (Dioscorea), and Bian Xu (Polygonum Aviculare).

22. A sedative herbal mixture in a suppository to calm a human and induce sleeping status, the herbal mixture comprising:
a. at least one herb selected from the group consisting of Fu-ling (Poria Cocos), Qian Shi (Euryale Ferox), Bi Xie (Dioscorea), Shan Yao (Dioscorea Batata) and Tian Huan Fen (Trichosanthes Kcrilowii);
b. at least one herb selected from the group consisting of Bing Pian (Dryobalanops Aromatica), Bo He (Mentha Arvensis) and Zhang Nao (Cinnamomum Camphora); and
c. at least one herb selected from the group consisting of Zhu Sha (Cinnabaris), Ci Shi (Magnetium), Suan Zao Ren (Zizyphus Sativavar), Bai Zi Ren (Biotae Orientalis Semen), Yuan Zhi (Polygala Tenuifolia), and He Huan Pi (Albizza Julivrissin).
